(19) United States
(12) Patent Application Publication
(10) Pub. No.: US 2021/0219846 A1
(43) Pub. Date: Jul. 22, 2021

(54) APPARATUS AND METHOD OF NON-INVASIVELY DETERMINING DEEP TISSUE TEMPERATURE USING MICROWAVE RADIOMETRY

(71) Applicant: Brain Temp, Inc., Bryn Mawr, PA (US)

(72) Inventor: Robert C. Allison, Rancho Palos Verdes, CA (US)

(73) Assignee: Brain Temp, Inc., Bryn Mawr, PA (US)

(21) Appl. No.: 17/153,360

(22) Filed: Jan. 20, 2021

Related U.S. Application Data
(60) Provisional application No. 62/963,578, filed on Jan. 21, 2020.

(51) Int. Cl. A61B 5/01; A61B 5/0507; A61B 5/00; G01K 11/00

(52) U.S. Cl. CPC A61B 5/01; A61B 5/0507; A61B 2562/0271; G01K 11/006; A61B 5/6833

(57) ABSTRACT

An apparatus for measuring a target tissue temperature is provided. The sensor antenna may include an outside and a contact side. A sensor antenna measurement aperture may be disposed on the contact side. The sensor antenna measurement aperture may be configured to generate a first signal. A skin temperature sensor may be disposed on the contact side and configured to generate a second signal. A radiometer may be configured to receive the first signal and the second signal.

Determining tissue temperature is desirable for diagnosis and treatment of numerous conditions. Conventional diagnostic methods and treatments utilize invasive methods of tissue measurement, which significantly increases risks and recovery, as well as costs. Thus, non-invasive methods of measuring temperature are desired. However, even if with non-invasive methods, it is difficult to ascertain the temperature at a specified location or depth.

In particular, cerebral temperature is a significant indicator in disruptions of important life functions. However, the cerebral temperature is largely inaccessible and therefore not easily measured. Additionally, a human's epidural temperature is lower than the temperature at the center of the brain. Moreover, the temperature of the surface of the brain differs from the temperature at the center of the brain. Thus, even an intrusive measurement at the surface of the brain may not be telling of the temperature at its core.

It would be desirable, therefore, to provide apparatuses, systems, and methods for determining tissue temperature at a specified depth or location, in a non-invasive manner.

### SUMMARY OF THE INVENTION

Disclosed herein are systems, apparatuses, and methods for determining temperature of tissue non-invasively.

In an embodiment, the invention of the present disclosure may be an apparatus for measuring a target tissue temperature comprising a sensor antenna having an outside and a contact side. In a further embodiment, the apparatus comprises a sensor antenna measurement aperture disposed on the contact side, where the sensor antenna measurement aperture is configured to generate a first signal. The invention of the present disclosure may further comprise a skin temperature sensor disposed on the contact side, where the skin temperature sensor is configured to generate a second signal. In an embodiment, the apparatus includes a radiometer configured to receive the first signal and the second signal, in electrical communication with the sensor antenna, the sensor antenna measurement aperture, and the skin temperature sensor. The target tissue temperature may be calculated via the equation T_{target}=Tₛₖᵢₙ+(T_{average}−Tₛₖᵢₙ)*c, where T_{target} is the target tissue temperature, Tₛₖᵢₙ is the patient's skin temperature, T_{average} is the average temperature, and c is a constant.

In further embodiments, the apparatus may include a remote switch module disposed between the sensor antenna and the radiometer. Moreover, in an embodiment, the constant may be determined experimentally based on a preexisting dataset. In even a further embodiment, the average temperature is a weighted average temperature. In such an embodiment, the weighted average temperature may be proportional to the summation of T_{d}*A*e^{(-d/c1)} from the patient's skin to the target tissue, where d is the variable depth of a tissue, T_{d} is the temperature at a depth d, A is a constant, and c1 is a constant. Thus, fractional contribution to the weighted average temperature (radiometer temperature) may be calculated from any particular depth.

In one embodiment, the apparatus may further include an isolator, a low noise amplifier, a band pass filter, a microwave detector, a video amp, a synchronous detector, and/or a low pass filter.

In an embodiment, the invention of the present disclosure is a method to measure a target tissue temperature comprising placing a sensor antenna on a patient's skin, where the sensor antenna has a sensor antenna measurement aperture and a skin temperature sensor. In an embodiment, the method may also include detecting, via the sensor antenna, a plurality of microwave emissions from a measurement volume of tissues, where the measurement volume of tissues comprises a plurality of tissue layers. The method may further include detecting, via the skin temperature sensor, a patient's skin temperature. Moreover, the method may include calculating an average temperature of the measurement volume of tissues; and calculating the target tissue temperature via the equation T_{target}=Tₛₖᵢₙ+(T_{average}−Tₛₖᵢₙ)*c, where T_{target} is the target tissue temperature, Tₛₖᵢₙ is the patient's skin temperature, T_{average} is the average temperature, and c is a constant.

In one embodiment, the constant may be determined experimentally based on a preexisting dataset. In another embodiment, the average temperature may be a weighted average temperature, calculated by weighing the average temperature based on an attenuation level of each of the plurality of tissue layers. In one embodiment, adhesive may be disposed on the sensor antenna.

### BRIEF DESCRIPTION OF THE DRAWINGS

FIG. 1 illustrates a plot of muscle conductivity versus frequency.

FIG. 2 illustrates a plot of temperature versus depth in live swine beginning from the surface and going to a depth within brain tissue

FIG. 3 illustrates a power loss density plot.

FIG. 4 illustrates a block diagram of an embodiment of the present invention having a radiometer.

FIG. 5 illustrates a block diagram of an embodiment of the present invention having a radiometer and a remote switch.

FIG. 6 illustrates an embodiment of the sensor antenna with switch component attached.

While the invention is described with reference to the above drawings, the drawings are intended to be illustrative, and the invention contemplates other embodiments within the spirit of the invention.

### DETAILED DESCRIPTION

The present invention will now be described more fully hereinafter with reference to the accompanying drawings which show, by way of illustration, specific embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as devices or methods. Accordingly, the present invention may take the form of an entirely hardware embodiment, an entirely software embodiment, or an embodiment combining software and hardware aspects. The following detailed description is, therefore, not to be taken in a limiting sense.

Throughout the specification and claims, the following terms take the meanings explicitly associated herein, unless the context clearly dictates otherwise. The phrases “in one embodiment,” “in an embodiment,” and the like, as used herein, does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” includes plural references. The meaning of “in” includes “in” and “on.”

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

Disclosed herein are devices, systems and methods (the “System”) for measurement of tissue temperature at varying depths. Devices and methods for determining temperature of tissue non-invasively are hereby provided.

In an embodiment, microwave emission resulting from thermal activity in body tissue may be used to discern the temperature of the tissue non-invasively. The depth beneath the skin from which the microwave emissions may be detected is primarily determined by tissue attenuation resulting from electrical conductivity in the tissue. This attenuation may be frequency dependent.

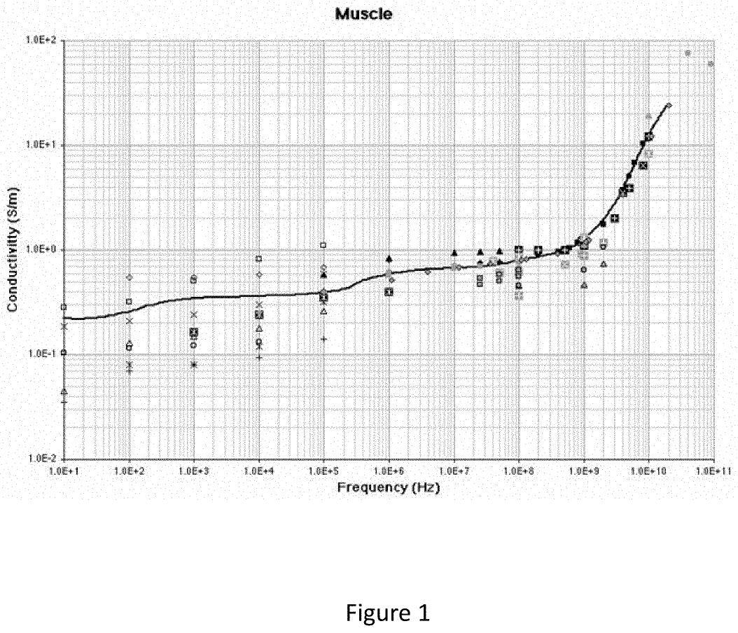

FIG. 1 illustrates a plot of muscle conductivity versus frequency. Referring to FIG. 1, the conductivity of the muscle is relatively constant up to roughly 1 GHz. As the frequency surpasses 1 GHz, the conductivity, and therefore the attenuation, begins to increase rapidly.

In an embodiment, a measurement from the skin surface may include the emissions from the total volume of tissue in the sensor reception area, out to a depth where attenuation decreases the magnitude of the emitted energy to an undetectable level. As a non-limiting example, the sensor reception area may be a 1″×1″ square disposed on the surface of a patient's skin. In such a non-limiting example, a non-filtered measurement of the emissions would include the sum of emissions from the most outer layer of tissue to the deepest layer that provides readable emission levels. Further, in such a non-limiting example, if the deepest readable layer were 2″ deep, the overall total volume of tissue in the reception area would be 2 cubic inches. However, in various embodiments the sensor reception area may be any area and the volume of tissue may be any volume.

In one embodiment, the frequencies where attenuation is lowest will include the deepest temperature contributions. By selecting appropriate detection frequencies, emissions can be measured that include contributions from differing depths. For example, depths of 12-14 millimeters, or any other suitable depth, may be measured.

In an embodiment, temperatures from two or more measurement volumes may be used to determine the temperature in the region where the volume is not common to both (all) measurements. Thus, temperature at a deep region beneath the skin surface may be determined using two temperature measurements. In an embodiment, one microwave measurement may be used, that includes temperature contributions from the region of interest and a skin temperature measurement. In further embodiments, any number of microwave measurements and/or any number of skin temperature measurements may be compounded, weighted, or otherwise used to determine temperature for a desired depth.

In an exemplary embodiment, brain tissue may be targeted. The user of the System may desire a temperature reading at a particular depth of the brain. Thus, temperature measurements at different depths (such as 1 millimeter, 2 millimeter, 4 millimeter, and any other suitable measurements) may be determined. According to an embodiment, a temperature gradient is determined between the deep brain temperature, and temperature at skin surface. However, in alternate embodiments, a temperature gradient may be determined between the deep brain temperature, and any other temperature measured at any region on the body.

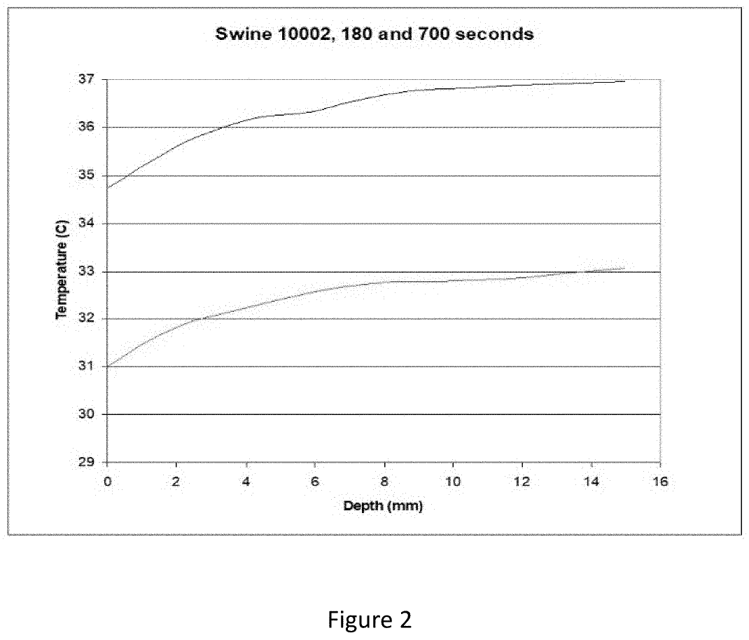

In one embodiment, the temperature gradient may be determined using one or more thermocouples, or an array of thermocouples, embedded within skulls of living pigs. However, in various embodiments, the temperature gradient may be collected from various sources. The temperature gradient between the inner temperature and surface temperate may be the result of blood profusion and normal heat flow to the surrounding environment. FIG. 2 illustrates a plot of temperature versus depth beginning from the surface and going to a depth within brain tissue. At a deeper depth, such as around 13 mm, the temperature asymptotically approaches a constant value. However, in alternate embodiments, the ratio between temperature and depth may vary at various depths.

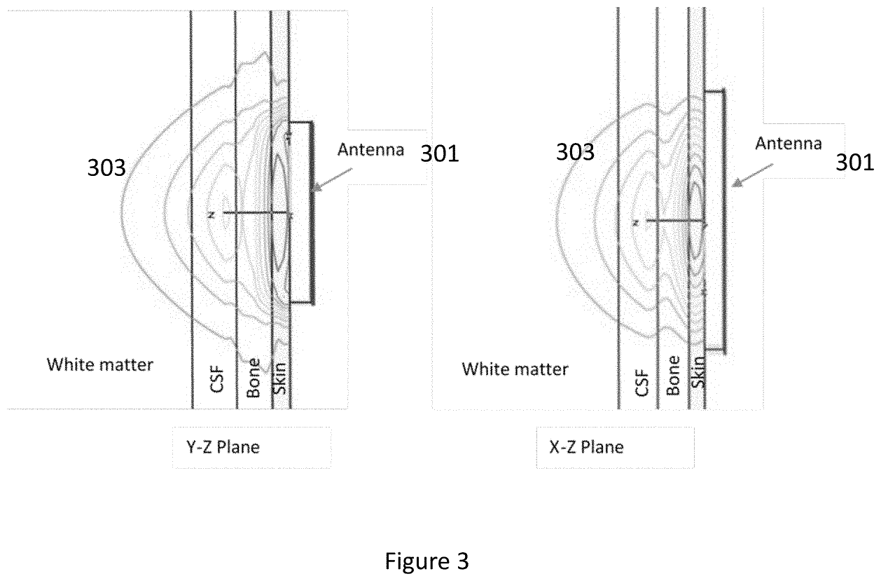

In an embodiment, a sensing antenna 301 is used to contact the skin. The sensing antenna 301 that is positioned in contact with the skin may have a reception pattern 303 described by the power loss density plot, as illustrated in FIG. 3.

In an embodiment, power loss density may be determined. The Principle of Reciprocity in Antenna Theory may be used to determine the power loss density. As a non-limiting example, the power loss density is determined as the reciprocal of the sensing pattern. In an embodiment, the power loss density of the microwave signal entering the tissue may describe the contribution from each point in the layers of tissue to the total power received by the antenna. In a further embodiment, the distribution of power loss density may be determined using 3D electromagnetic simulation software. In such an embodiment, the power distribution may be a curve fit to an equation describing the contribution as a function of depth into the layers of tissue. As a non-limiting example, this equation may be: fractional contribution of total received power as a function of depth=A*e^{(-depth/C)}, where A and C are constants.

In an embodiment, energy received by the sensor may be determined as a weighted average of the emissions from temperatures within the measurement volume. That is, total received signals from emissions may be shaped by the attenuation in the volume. Emissions from distant tissue may be attenuated so temperature may be weighted towards the closer tissue. However, in an embodiment, the user may tailor the weighted average of the emissions so the temperature may be weighted towards any layer of tissue. In another embodiment, the device may be configured to account for the weighted average without need for the user to intervene or adjust settings.

In an embodiment, the radiometer input received from the antenna is proportional to a summation over all depths of the fraction of signal power received from each layer of depth multiplied by the temperature at that depth. As a non-limiting example, the radiometer temperature may be represented by the equation:

$$\begin{matrix}Radiometer \\ Temperature\end{matrix} = {\sum }_{d = 0}^{d > 4 cm} \{ \begin{matrix}Temperature \\ at depth d\end{matrix} \times \begin{matrix}Fractional contribution \\ at depth d\end{matrix} \}$$

In an embodiment, the fractional contribution of total received power as a function of depth may be represented by the equation: A*e^{(-depth/C)}, where A and C are constants. However, any variation of equations may be used to represent the radiometer temperature and/or the fractional contribution.

In further embodiments, the apparatus may include a remote switch module disposed between the sensor antenna and the radiometer. Moreover, in an embodiment, the constant may be determined experimentally based on a preexisting dataset. In even a further embodiment, the average temperature is a weighted average temperature. In such an embodiment, the weighted average temperature may be proportional to the summation of T_{d}*A*e^{(-d/c1)} from the patient's skin to the target tissue, where d is the variable depth of a tissue, T_{d} is the temperature at a depth d, A is a constant, and c1 is a constant. Thus, fractional contribution to the weighted average temperature (radiometer temperature) may be calculated from any particular depth, and multiplied by the temperature at the particular depth. The depths may then be summed.

In an embodiment, brain temperature at a deep depth may be determined by determining an average temperature in a volume that includes both deep brain temperature and a temperature at one end of the temperature gradient curve. In a further embodiment, the average temperature and the temperature at one end (such as the skin), may be used to determine the temperature at the other end (deep depth temperature). At certain depths, temperature may be nearly constant. In one embodiment, a straight line may be substituted between the end point temperatures for the weighted average temperature curve. In an alternate embodiment, a line of best fit (including a curved line) may be substituted between any temperatures for the weighted average temperature curve. An exemplary calculation for determining deep brain temperature may be as follows:

T_{brain}=Tₛₖᵢₙ+(T_{average}−Tₛₖᵢₙ)×2

In an embodiment where a weighted average temperature is used, the constant value of “2” may be changed, depending on microwave tissue properties and geometry. In another embodiment, using 3D EM simulation software, the constant may be determined by calculating the power loss density in the measurement volume, multiplying each point in the volume by the temperature at that point, and integrating over the entire volume to find the weighted average temperature. Alternatively, a constant may be calculated by experimentally determining the constant using live animal measurements. However, in alternate embodiments, the constant may be calculated using any combination of theoretical, calculated, hypothesized, or experimental data sets.

In another embodiment, the aforementioned constant may be the Head Factor (HF). In such an embodiment, the HF may be a function of the thermal properties of the skull or other layer and the microwave tissue properties within the skull or other tissue. In an embodiment, the initial value was determined from an animal trial measurement and from electromagnetic simulations using published values on microwave tissue properties.

Therefore, in accordance with the invention, a microwave antenna may be used for determining deep tissue temperature in the brain, at a specified depth, and implement the methods above. The microwave antenna may include a thermistor for measuring skin surface temperature. The microwave antenna or System may further be in communication with an external monitor or computer. In alternative embodiments, there may be more than one microwave antennas or more than one sensors. In further embodiments, the device may include any number or combination of processors, memory units, electronic storage devices, or other electronic components.

In an embodiment, selecting an operating frequency band where potentially interfering devices are not allowed to operate may mitigate unwanted microwave noise. Further, the antenna aperture may be shielded from external sources. In such an embodiment, the shielding may involve configuring the antenna such that outside interference has to propagate through enough tissue layers before reaching the aperture, allowing the unwanted signal to diminish to undetectable levels.

In an embodiment, the magnitude of the microwave emissions collected from a portion of tissue by the sensor antenna is converted to a temperature indication by a microwave radiometer. In an embodiment, the radiometer measurement frequency is selected for measurement depth and other practical considerations such as antenna size and avoidance of potentially interfering electronic devices.

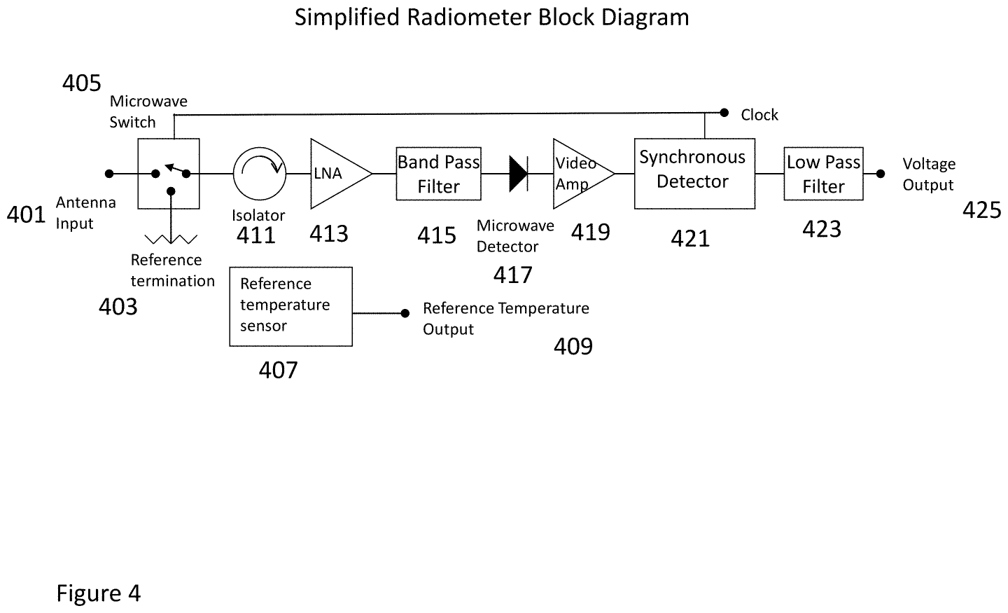

FIG. 4 illustrates an embodiment of a radiometer block diagram 400. In an embodiment, a microwave switch 405 alternately selects between the antenna input 401 and a reference termination 403 of known temperature at a 50% duty factor clock rate, which may enable the use of synchronous detection during signal processing. In a further embodiment, a temperature sensor 407 is located adjacent to a reference termination 403 and measures the reference temperature. In an embodiment, the signal next is passed through an isolator 411. In an embodiment, the switch output is amplified by a low noise amplifier 413 and is filtered (for example, through a band pass filter 415). Further, a microwave detector 417 may detect the modulation created by the switch 405.

In an embodiment, a video amp 419 may be positioned after the microwave detector 417. The video amp 419 may be a low frequency AC amplifier. The video amp 419 may be configured to amplify the detector output voltage, which may be 100 Hz but may be higher (for example, 1 Khz or 10 Khz). The video amp 419 may allow for no DC component of a signal to pass. The frequency threshold of the video amp 419 may be set in relation to the switch modulation rate. The modulation may then be filtered and rectified by a synchronous detector 421. In an embodiment, the output is low pass filtered via a low pass filter 423, resulting in a DC voltage 425 proportional to the temperature difference between the antenna input 401 from the head and the reference termination 403. The temperature difference may be added to the reference temperature sensor output 409, resulting in the radiometer temperature.

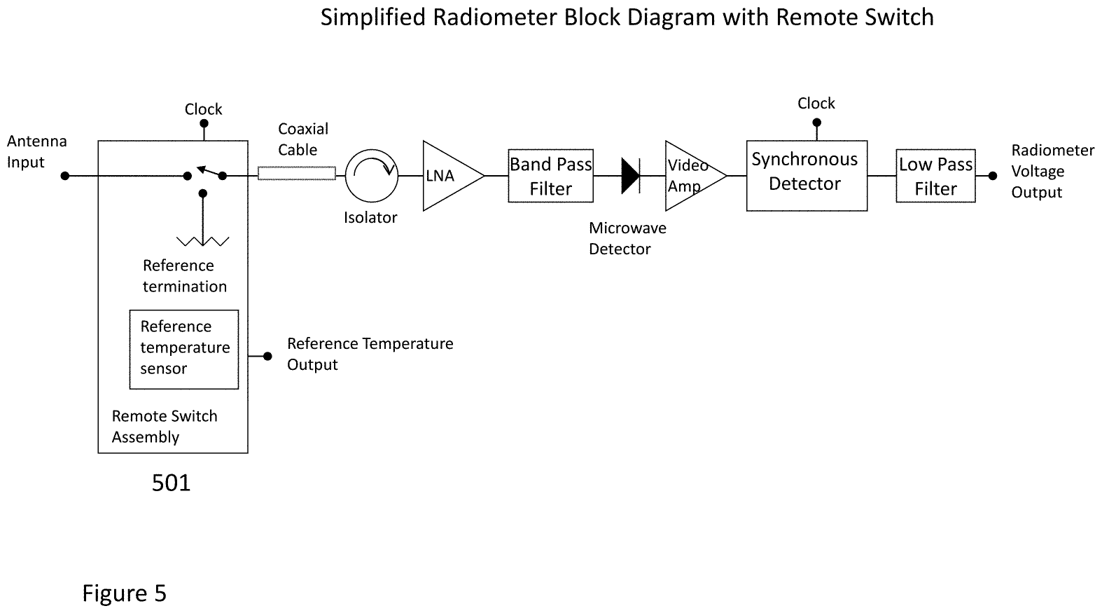

FIG. 5 illustrates an embodiment of a radiometer block diagram 500 including a remote switch assembly 501. In such an embodiment, the remote antenna may provide for convenience and comfort to the patient. As a non-limiting example, in the remotely located antenna and switch embodiment, the bulk of the weight of the radiometer is not hanging on the patient's skin. The remotely located switch may also minimize the temperature errors introduced by the coaxial cable that separates the antenna and switch from the radiometer housing.

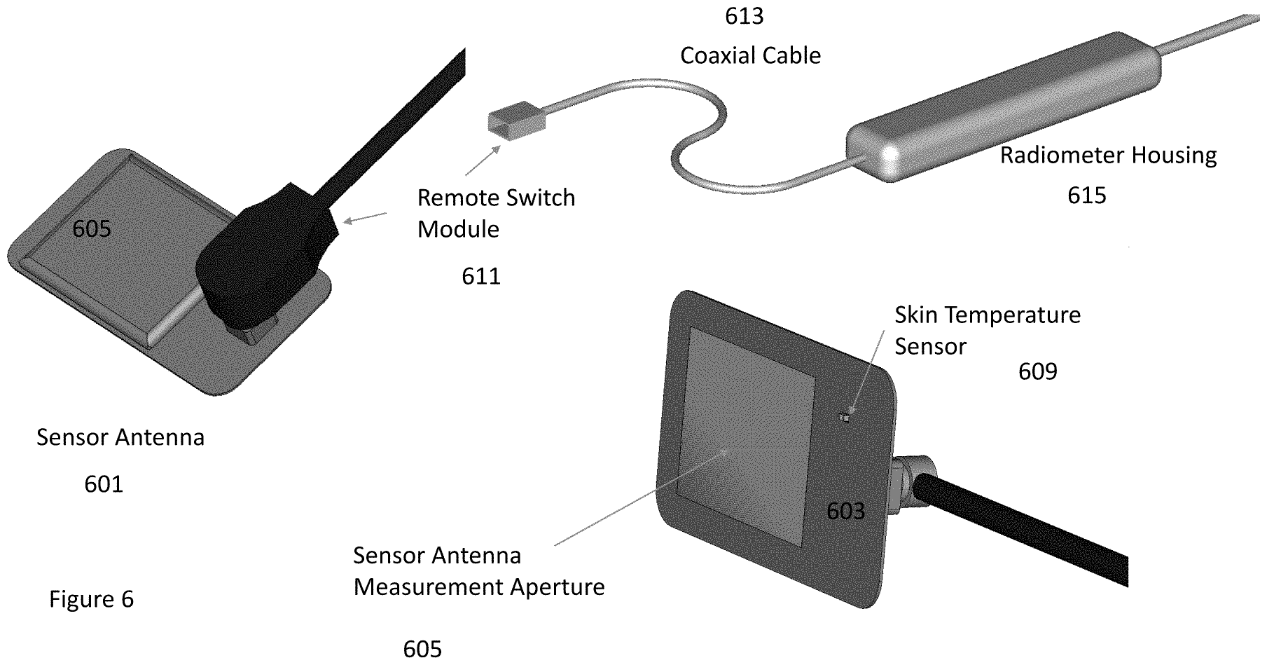

FIG. 6 illustrates an embodiment of the sensor antenna 601 including the switch component 611. In such an embodiment, the antenna 601 is a separable, disposable item. In a further embodiment, the face of the adhesively attached antenna 601 may include the receiving aperture 605 and a skin temperature sensor 609. The skin temperature sensor 609 may be a thermistor, thermocouple, or other small temperature measurement device. In alternative embodiments, any number or type of components may be disposed on the remote switch module 611.

The sensor antenna 601 may have a contact side 603 and an outside 605. The contact side 603 may be configured to index with a patient's skin. The outside 605 may face away from the patient. The contact side 603 of the sensor antenna 601 may be coated with an adhesive, such that the sensor antenna 601 adheres to the patient's skin. However, in alternate embodiments, the sensor antenna 601 may be held in place with any number of methods. In an embodiment, the contact side 603 of the sensor antenna 601 may include a sensor antenna measurement aperture 605 and/or a skin temperature sensor 609.

In an embodiment, the sensor antenna is connected to the remote switch module 611. In an embodiment the remote switch module 611 is further connected to the radiometer housing 615 utilizing a coaxial cable 613. However, in alternate embodiments, any number of electronic communications tethers may be used. In yet further embodiments, any suitable form of low-loss microwave transmission lines may be used.

While this invention has been described in conjunction with the embodiments outlined above, many alternatives, modifications and variations will be apparent to those skilled in the art upon reading the foregoing disclosure. Accordingly, the embodiments of the invention, as set forth above, are intended to be illustrative, not limiting. Various changes may be made without departing from the spirit and scope of the invention.

What is claimed is:

1. An apparatus for measuring a target tissue temperature comprising:
a sensor antenna including an outside and a contact side;
a sensor antenna measurement aperture disposed on the contact side, the sensor antenna measurement aperture configured to generate a first signal;
a skin temperature sensor disposed on the contact side, the skin temperature sensor configured to generate a second signal; and
a radiometer, configured to receive the first signal and the second signal, in electrical communication with the sensor antenna, the sensor antenna measurement aperture, and the skin temperature sensor,
wherein the target tissue temperature is calculated via the equation: T_{target}=Tₛₖᵢₙ+(T_{average}−Tₛₖᵢₙ)*c,
wherein T_{target} is the target tissue temperature, Tₛₖᵢₙ is the patient's skin temperature, T_{average} is the average temperature as measured by the radiometer, and c is a constant.

2. The apparatus of claim 1, further comprising a remote switch module disposed between the sensor antenna and the radiometer.

3. The apparatus of claim 1, wherein the constant (c) is determined experimentally based on a preexisting dataset.

4. The apparatus of claim 1, wherein the average temperature is a weighted average temperature.

5. The apparatus of claim 4, wherein the weighted average temperature is proportional to the summation of T_{d}*A*e^{(-d/c1)} from the patient's skin to the target tissue, where d is the variable depth of a tissue, T_{d} is the temperature at a depth d, A is a constant, c1 is a constant.

6. The apparatus of claim 1, further comprising an isolator, a low noise amplifier, a band pass filter, a microwave detector, a video amp, a synchronous detector, and a low pass filter.

7. A method to measure a target tissue temperature comprising:
disposing a sensor antenna on a patient's skin, the sensor antenna including a sensor antenna measurement aperture and a skin temperature sensor;
detecting, via the sensor antenna, a plurality of microwave emissions from a measurement volume of tissues, the measurement volume of tissues comprising a plurality of tissue layers;
detecting, via the skin temperature sensor, a patient's skin temperature;
calculating an average temperature of the measurement volume of tissues; and
calculating the target tissue temperature via the equation:
T_{target}=Tₛₖᵢₙ+(T_{average}−Tₛₖᵢₙ)*c,
wherein T_{target} is the target tissue temperature, Tₛₖᵢₙ is the patient's skin temperature, T_{average} is the average temperature, and c is a constant.

8. The method of claim 7, wherein the constant is determined experimentally based on a preexisting dataset.

9. The method of claim 7, wherein the average temperature is a weighted average temperature, calculated by weighing the average temperature based on an attenuation level of each of the plurality of tissue layers.

10. The method of claim 7, wherein an adhesive is disposed on the sensor antenna.
